(19) United States
(12) Patent Application Publication
(10) Pub. No.: US 2009/0259243 A1
(43) Pub. Date: Oct. 15, 2009

(54) POWER SUPPLY APPARATUS FOR OPERATION

(76) Inventors: Naoko Tahara, Hachioji-shi (JP); Koh Shimizu, Kodaira-shi (JP)

Correspondence Address: OSTROLENK FABER GERB & SOFFEN, 1180 AVENUE OF THE AMERICAS, NEW YORK, NY, 100368403, US

(21) Appl. No.: 12/103,024

(22) Filed: Apr. 15, 2008

(51) Int. Cl. A61B 17/32

(52) U.S. Cl. 606/169

(57) ABSTRACT

A power supply apparatus for operation for outputting power to a surgical instrument includes a phase difference detection section for detecting a phase difference between an output voltage and an output current in the output, and an abnormality detection section for detecting an abnormality according to whether or not a period of time for which the phase difference deviates from a predetermined normal value range exceeds a predetermined period of time. Further, a power supply apparatus for operation for outputting power to a surgical instrument includes a phase difference detection section for detecting a phase difference between an output voltage and an output current from the power in the output, and an abnormality detection section for detecting an abnormality according to whether or not a variation value of the phase difference per unit time exceeds a predetermined threshold. By detecting the abnormality in this manner, the surgical instrument can be prevented from being broken.

### BACKGROUND OF THE INVENTION

1. Field of the Invention

The present invention relates to a power supply apparatus for operation.

2. Description of the Related Art

A drive apparatus for an ultrasonic vibrator is hitherto known as a power supply apparatus for operation. For example, in Jpn. Pat. Appln. KOKAI Publication No. 2004-267332, a drive apparatus employing a phase-locked loop (PLL) system in which control is performed in such a manner that a drive frequency of an ultrasonic vibrator coincides with a resonant frequency is disclosed. Further, in Jpn. Pat. Appln. KOKAI Publication No. 2002-209907, an ultrasonic operation system which can maintain resonance of a converter even when a load or a change in temperature that varies the resonant frequency exists is disclosed.

### BRIEF SUMMARY OF THE INVENTION

A first aspect of the present invention relates to a power supply apparatus for operation for outputting power to a surgical instrument, the apparatus comprising: a phase difference detection section for detecting a phase difference between an output voltage and an output current from the power in the output; and an abnormality detection section for detecting an abnormality according to whether or not a period of time for which the phase difference deviates from a predetermined normal value range exceeds a predetermined period of time.

Further, a second aspect of the present invention relates to a power supply apparatus for operation for outputting power to a surgical instrument, the apparatus comprising: a phase difference detection section for detecting a phase difference between an output voltage and an output current from the power in the output; and an abnormality detection section for detecting an abnormality according to whether or not a variation value of the phase difference per unit time exceeds a predetermined threshold.

Further, a third aspect of the present invention relates to the first aspect, and the predetermined period of time is 100 msec or more.

Further, a fourth aspect of the present invention relates to the second aspect, and the predetermined threshold per unit time is 10 degrees/100 msec.

Furthermore, a fifth aspect of the present invention relates to the first or second aspect, and when an abnormality is detected by the abnormality detection section, the output of the power to the surgical instrument is stopped.

Moreover, a sixth aspect of the present invention relates to the first or second aspect, and the surgical instrument is provided with an ultrasonic vibrator and a probe for transmitting the vibration of the ultrasonic vibrator to a distal end thereof, and the power to be output is ultrasonic power for driving the ultrasonic vibrator.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the preferred embodiments given below, serve to explain the principles of the invention.

FIG. 1 is an external perspective view of an ultrasonic operation system.

FIG. 2 is a view showing a schematic configuration of the ultrasonic operation system.

FIG. 3 is a view showing a state where a drive current generated in an ultrasonic power source unit flows to the hand-piece side.

FIG. 4 is a view showing a relationship between a voltage phase and a current phase.

FIG. 5 is a view for explaining a procedure for scanning for a resonant frequency fr.

(A) in FIG. 6 is a view showing a probe part in an enlarging manner.

(B) and (C) in FIG. 6 are graphs showing frequency dependence of the impedance Z, current I, and phase difference (θV−θI) which are under the PLL control observed when a crack develops in a probe in a normal state.

FIG. 7 is a functional block diagram for explaining a function of each unit in an ultrasonic power source unit in an ultrasonic operation system.

FIG. 8 is a flowchart for detecting an abnormality of a probe according to a first embodiment.

FIG. 9 is a graph showing time dependence of a phase difference (θV−θI) for explaining a second embodiment.

FIG. 10 is a flowchart for detecting an abnormality of a probe according to a third embodiment.

FIG. 11 is a functional block diagram for explaining a function of each unit in the ultrasonic power source unit in an ultrasonic operation system according to a fourth embodiment.

### DETAILED DESCRIPTION OF THE INVENTION

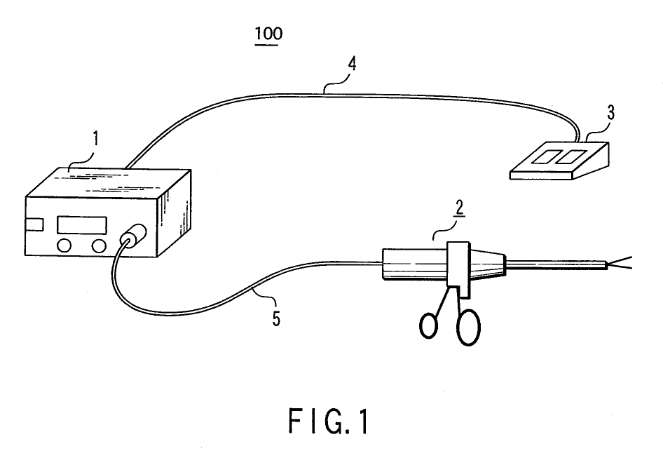

Embodiments of the present invention will be described below in detail with reference to the accompanying drawings. An endoscopic surgical operation for performing medical treatment of a diseased part to be performed by using a scope for observing a state in an abdominal cavity of a patient is known. FIG. 1 is an external perspective view of an ultrasonic operation system used as an example of a system for such an endoscopic surgical operation. The ultrasonic operation system is constituted of an ultrasonic power source unit 1 serving as a power supply apparatus for operation for generating an ultrasonic output for driving an ultrasonic vibrator, a hand-piece 2 serving as an ultrasonic surgical instrument for performing treatment by using an ultrasonic output supplied from the ultrasonic power source unit 1 through a cable 5, and a foot switch 3 connected to the ultrasonic power source unit 1 through a cable 4, for controlling the ultrasonic output from the ultrasonic power source unit 1.

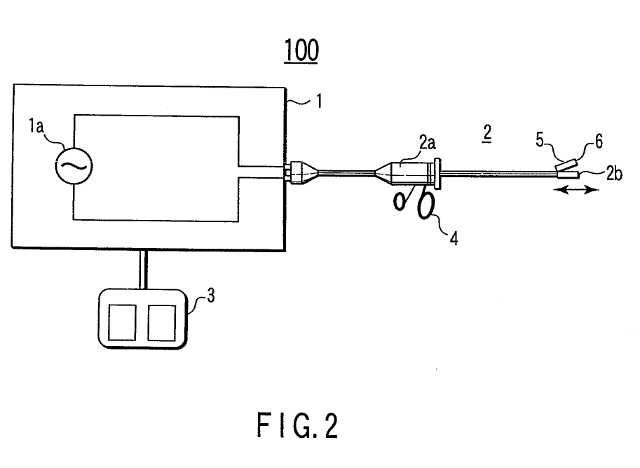

In FIG. 2, the hand-piece 2 is constituted of a hand-piece main body section 2a which includes handles 4, and in which an ultrasonic vibrator (not shown) is incorporated, and a probe 2b for transmitting vibration of the ultrasonic vibrator to a treatment section 5. The ultrasonic power source unit 1 is provided with an ultrasonic oscillator circuit 1a for generating electric energy for vibrating the ultrasonic vibrator. An electric signal output from the ultrasonic power source unit 1 is converted into mechanical vibration (ultrasonic vibration) by the ultrasonic vibrator inside the hand-piece main body section 2a, and thereafter the vibration is transmitted by the probe 2b to the treatment section 5. The treatment section 5 is provided with a grasping section 6 called a jaw driven to be opened or closed with respect to the distal end of the probe 2b. When the handles 4 are operated, the grasping section 6 is driven to be opened or closed with respect to the distal end of the probe 2b, and coagulation or incision of living tissue is performed by utilizing frictional heat generated by holding the living tissue between the distal end of the probe 2b and the grasping section 6 and applying the ultrasonic vibration thereto.

In this probe 2b, a crack is caused due to a scratch received when the probe 2b comes into contact with forceps or a clip during an operation. When a crack is caused to the probe 2b during an operation, it is necessary to immediately stop ultrasonic vibration, and replace the probe with a new one. If the operation is continued in the state where the crack is caused to the probe, it is conceivable that there is the possibility of the probe part being broken and falling off. Accordingly, it becomes necessary to detect the occurrence of the crack at an early stage, and inform the medical pursuer of the occurrence of the crack. The ultrasonic operation system will be described below in detail, and an apparatus and a method for exactly detecting an occurrence of a crack in a probe in an early stage will be described.

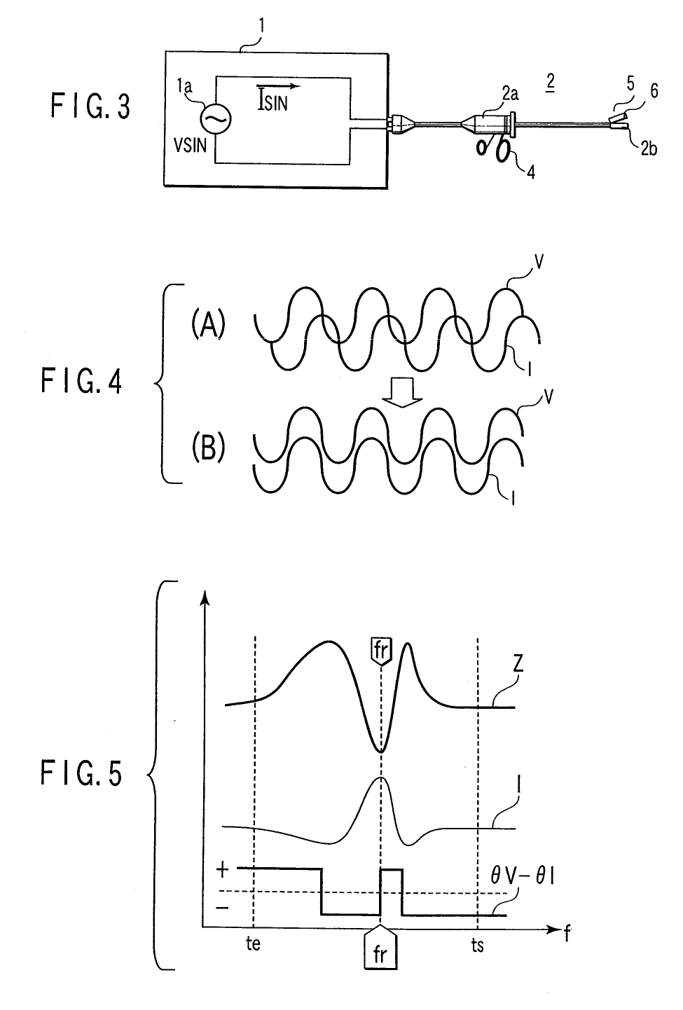

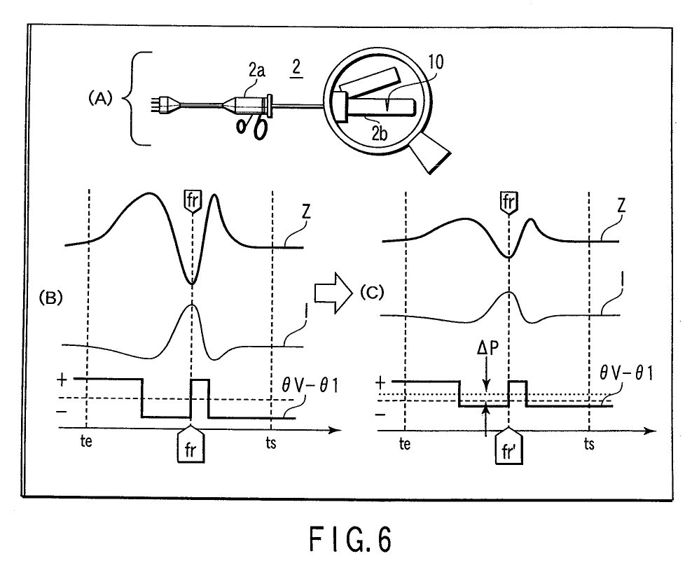

FIGS. 3 to 5 are views for explaining a method of controlling ultrasonic drive in an ultrasonic operation system. In FIG. 3, in an ultrasonic oscillator circuit la, a sinusoidal drive voltage VSIN is generated. When a sinusoidal drive current ISIN corresponding to the sinusoidal drive voltage VSIN flows into the ultrasonic vibrator inside the hand-piece main body section 2a, the ultrasonic vibrator converts the electric signal into mechanical vibration, and transmits the mechanical vibration to the distal end of the probe 2b. In the ultrasonic drive described above, when the ultrasonic vibration is output at a constant oscillation frequency, a phase difference occurs between the voltage V and the current I, and hence the drive efficiency lowers. Thus, a control circuit is provided in the ultrasonic power source unit 1, and the drive of the ultrasonic vibrator is performed while a resonance point at which a phase difference between the voltage V and the current I becomes 0 ((B) in FIG. 4) is searched for.

For example, in FIG. 5, the abscissa indicates the frequency f, and the ordinate indicates the impedance Z, current I, and phase difference (θV−θI). A value (θV−θI) indicates a phase difference. In this embodiment, a resonant frequency fr at which the phase difference (θV−θI) becomes 0 is detected by scanning for a point at which the impedance Z is minimized while consecutively changing the frequency. The control circuit 1c starts to perform the drive of the ultrasonic vibrator at the detected resonant frequency fr.

### First Embodiment

(A) to (C) in FIG. 6 are views for explaining a method of investigating an abnormality of a hand-piece 2 according to a first embodiment. (A) in FIG. 6 is a view showing a probe 2b part of the hand-piece 2 in an enlarging manner. This view schematically shows a state where the probe 2b has a crack 10. Here, the term crack does not necessarily imply a crack that can be confirmed with the naked eye, and includes a crack that does not appear externally, such as an internal crack, and a microcrack that appears at the early stage of metal fatigue. In the actual crack measurement, not only megascopic observation, but also microscopic observation using a magnifying glass, a metallurgical microscope or the like, and observation of a crack (microcrack) in the order of microns using an electron microscope are performed.

Measurement was conducted in detail so as to observe what variation occurs in the impedance Z and the phase difference (θV−θI) until a normal probe is cracked. The results are shown below.

(B) and (C) in FIG. 6 are graphs showing frequency dependence of the impedance Z, current I, and phase difference (θV−θI) which are under the PLL control observed when a crack has developed in the probe 2b in the normal state. At (B) in FIG. 6, the probe is not yet damaged, and the impedance Z, current I, and phase difference (θV−θI) which are in the normal state are shown. The frequency is varied by the PLL control such that the phase difference (θV−θI) becomes zero degree. (B) in FIG. 6, the phase difference (θV−θI) becomes also zero degree in the vicinity of a frequency at which the impedance Z becomes the lowest. Accordingly, this frequency fr is the resonant frequency.

(C) in FIG. 6 shows a graph of the impedance Z, current I, and phase difference (θV−θI) under the PLL control observed after the probe 2b is cracked. When the crack develops in the probe 2b, it is conceivable that the phase difference (θV−θI) is shifted, and the impedance is also largely varied. Further, the PLL control is performed such that the impedance becomes the minimum, and a new resonant frequency fr′ is searched for. (C) in FIG. 6 shows the impedance Z, current I, and phase difference (θV−θI) observed after the search, and it can be seen that the control is performed such that the phase difference (θV−θI) becomes in the vicinity of zero at the new resonant frequency fr′. However, it can also be seen that the minimum value of the impedance Z is larger than that at (B) in FIG. 6, and the value of the phase difference (θV−θI) is also at a value (dotted line) higher than the zero value (broken line) before the occurrence of the crack by ΔP. In the illustration of the phase difference (θV−θI) at (B) and (C) in FIG. 6, the degree of the positive/negative magnitude, and the polarities are shown schematically and rectangularly only for easy understanding. The characters ΔP indicating the variation in the phase difference (θV−θI) can also be produced by factors other than the crack in the probe. However, the value is several degrees or less, and a variation exceeding 10 degrees is attributable to a crack.

Even when the PLL control is performed, the impedance Z is varied by the crack produced in the probe 2b. It is conceivable that the impedance of the entire probe 2b has been varied, whereby the frequency characteristic of the impedance has been varied, and the frequency dependence of the phase difference (θV−θI) between the current and the voltage has also been varied. More specifically, the reason why the value of the phase difference (θV−θI) exhibits a value higher than before by ΔP can be conceivable that the probe 2b cannot sufficiently exhibit the function of the probe serving as a complete vibration transmitting element of the ultrasonic vibrator due to the crack, and another interference mode resulting from the crack is mixed with the vibration.

On the basis of these results, and by paying attention to the impedance Z of the hand-piece 2 under the PLL control, it is possible to measure the fact that a crack has been produced in the probe 2b by monitoring the variation with time in the phase difference (θV−θI) between a voltage phase signal θV and a current phase signal θI.

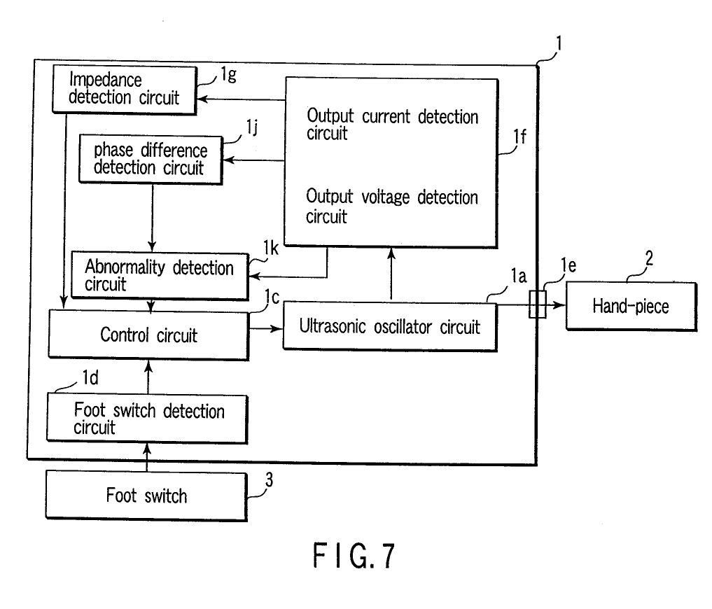

FIG. 7 is a functional block diagram for explaining a function of each unit in an ultrasonic power source unit in an ultrasonic operation system. The hand-piece 2 is connected to the ultrasonic power source unit 1 through a connector 1e. In the ultrasonic power source unit 1, an ultrasonic oscillator circuit 1a, output voltage/output current detection circuit 1f, impedance detection circuit 1g, phase difference detection circuit 1j, foot switch detection circuit 1d, and control circuit 1c are provided. The ultrasonic oscillator circuit 1a is a part for generating a drive signal for driving the ultrasonic vibrator inside the hand-piece 2. The foot switch detection circuit 1d is a part for detecting that the foot switch 3 has been operated by the operator.

When the foot switch 3 is operated by the operator, the operation signal is transmitted to the control circuit 1c through the foot switch detection circuit 1d. The control circuit 1c performs control such that the ultrasonic power is output from the ultrasonic oscillator circuit 1a to the hand-piece 2.

The output voltage/output current detection circuit 1f is a part for detecting an output voltage and an output current of the power supplied from the ultrasonic oscillator circuit 1a to the ultrasonic vibrator. The values of the output voltage and the output current detected by the output voltage/output current detection circuit 1f are input to the impedance detection circuit 1g and the phase difference detection circuit 1j. The impedance detection circuit 1g detects the impedance by using the impedance detection algorithm of the hand-piece 2 on the basis of the values of the input output voltage and the input output current, and the phase difference between them.

The phase difference detection circuit 1j detects, from the output voltage and the output current detected by the output voltage/output current detection circuit 1f, their phases (θV, θI) and the phase difference (θV−θI) between them.

The abnormality detection circuit 1k chronologically stores the value of the phase difference (θV−θI) transmitted from the phase difference detection circuit 1j in the internal storage part. More specifically, the value of the phase difference (θV−θI) is saved in a memory which is the storage part at intervals of unit time of, for example, 5 msec, and the consecutively measured value of the phase difference (θV−θI) and the previously saved value of the phase difference (θV−θI) are compared with each other. Further, the value of the phase difference (θV−θI) measured at intervals of 5 msec is compared with plural values of the phase difference (θV−θI) such as values measured 5 msec ago, 10 msec ago, 15 msec ago, and so on, thereby judging whether or not the value of the phase difference (θV−θI) is normal.

As a judging method, a case where the value of the phase difference (θV−θI) deviates from the normal value range over a certain fixed time is judged to be abnormal. As a result of an experiment, specifically, it has been found that when the value of the phase difference (θV−θI) is within ±10 degrees, i.e., when the absolute value of the phase difference (θV−θI) is 10 degrees or less, the probe 2b is not cracked, and the value is a normal value. When the absolute value of the phase difference (θV−θI) exceeds 10 degrees, the phase difference is in a state where it deviates from the normal value range, and here the state is defined as a state where the phase continues to lead or lag. Further, when the absolute value of the phase difference (θV−θI) exceeds 10 degrees, i.e., when there is a phase difference in the state where the phase continues to lead or lag, and if the period in which the above phase difference is present exceeds 100 msec, a crack that can be visually observed, or a microcrack that can be confirmed by an electron microscope develops. On the other hand, when the above period is 100 msec or less, a crack have hardly been confirmed visually or even by using an electron microscope.

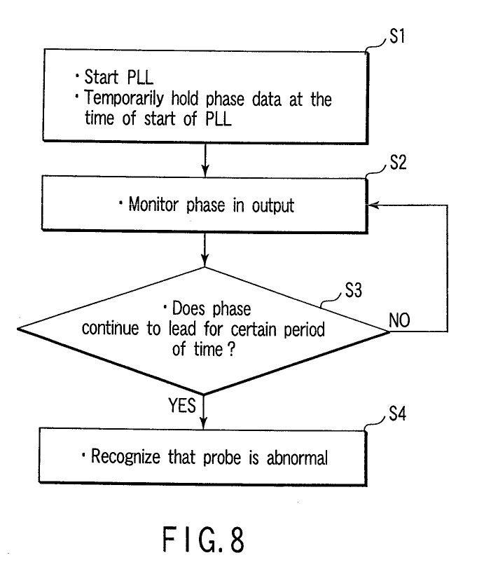

The above flow will be described below by using the flowchart of FIG. 8. First, when an operation in an abdominal cavity of a patient is performed by using an ultrasonic probe 2b, the control circuit 1c starts the PLL control, and the abnormality detection circuit 1k detects the initial voltage phase signal θV of the hand-piece 2, current phase signal θI, and phase difference (θV−θI) between them as phase data, and stores the detected data (step S1). The PLL control is the control necessary for the ultrasonic probe to perform an operation with increased energy efficiency. While the ultrasonic power is output from the ultrasonic oscillator circuit 1a to the hand-piece 2, the abnormality detection circuit 1k monitors the variation in the voltage phase signal θV, current phase signal θI, and phase difference (θV−θI) at intervals of a fixed sampling time determined in advance (step S2). The monitored phase data is compared with a plurality of phase data items detected previously. For example, the abnormality detection circuit 1k determines to set the sampling time at 5 msec, and compares each of 20 samples of the phase difference (θV−θI) (phase difference (θV−θI) values within a period of 5 msec×20 samples=100 msec) detected previously, or an average value of the 20 samples of the phase difference (θV−θI) detected previously with a currently detected phase difference (θV−θI). The abnormality detection circuit 1k compares the phase difference (θV−θI) with a predetermined value in the normal value range, for example, 10 degrees (in this case, 10 degrees implies ±10 degrees, and also implies whether the phase difference exceeds +10 degrees or is less than −10 degrees. That is, it is meant that the absolute value of the phase difference (θV−θI) is compared with this 10 degrees) to judge whether or not the phase difference deviates from the normal value range, i.e., whether or not the phase continues to lead or lag (step S3). When it is judged by the abnormality detection section 1k that the phase continues to lead or lag, it is recognized that probe abnormality has occurred (step S4). When the phase difference (θV−θI) is within the normal value range, the abnormality detection circuit lk judges that the probe 2b is normal, and returns to step S2 to continue monitoring the variation in the phase difference (θV−θI).

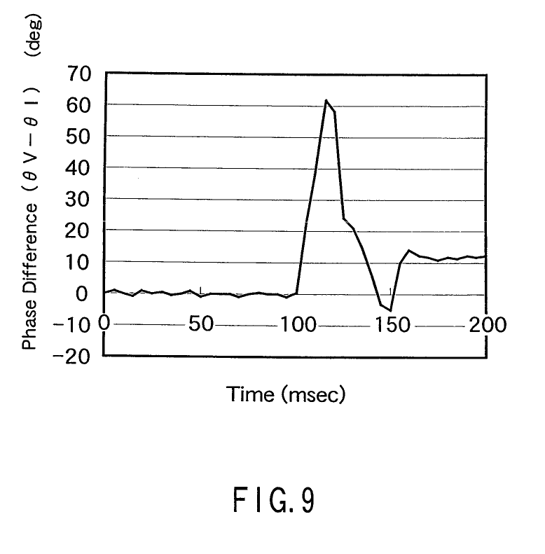

A part (corresponding to 200 msec) of the results obtained by continuously performing the measurement and by setting the sampling time at 5 msec are shown in FIG. 9 with the values of the actually measured phase difference (θV−θI) shown on the ordinate. It can be seen that the phase difference (θV−θI) between the output voltage and the output current varies at approximately 100 msec. More specifically, the phase difference abruptly increases, i.e., the phase difference varies from 0.5 degrees to 62.0 degrees between the sampling of 110 msec and sampling of 115 msec. After the phase difference abruptly changes, the phase difference (θV−θI) passes 0 degree between 140 msec and 145 msec, thereafter lowers to −5.0 degree at 150 msec, and then maintained at the level near 13 degrees again. The reason why the abruptly increased phase difference (θV−θI) thereafter lowers can be conceived that the cracked probe is subjected to frequency rescanning by the PLL control so as to further find lower impedance, whereby the shift to the resonance point position of the cracked probe has occurred. Although the phase difference (θV−θI) is stable at around 13 degrees, the probe is already cracked. Accordingly, if the probe is further used continuously, there is the possibility of the probe being broken, and falling off in the abdominal cavity of the patient. Accordingly, the abnormality detection circuit 1k transmits a signal to the control circuit 1c to cause the control circuit 1c to stop or shut down the ultrasonic output, to thereby prevent the probe from being broken and falling off. Further, the abnormality detection circuit 1k may display a warning so as to inform the operator of the crack developing in the probe.

### (Effect)

According to this embodiment, the phase difference (θV−θI) between the output voltage and the output current is detected, the variation value of the phase difference (θV−θI) is chronologically monitored, a normal range of the phase difference (θV−θI) is determined in advance, and when the state where the phase difference (θV−θI) deviates from the normal range over a predetermined period, the phase difference is detected as an abnormality, whereby it is possible to instantaneously and easily grasp an occurrence of a crack in the probe. By virtue of the detection of the probe crack in the early stage, the medical staff can replace the probe before the breakage of the probe occurs, and safely continue the treatment of the patient.

### Second Embodiment

A second embodiment of the present invention will be described below. As a judging method different from the first embodiment, it is possible to set, for example, a threshold determined in advance with respect to a value of a variation amount of a phase difference (θV−θI) in an abnormality detection circuit 1k. The abnormality detection circuit calculates a variation amount of the value of the phase difference (θV−θI) transmitted from a phase difference detection circuit 1j per unit time, compares the calculated variation amount with the predetermined threshold, and judges that the probe is abnormal when the variation amount exceeds the threshold. Here, how to determine the threshold will be described below with reference to the data of FIG. 9. FIG. 9 shows the phase difference (θV−θI) obtained by performing sampling at intervals of 5 msec by the phase difference detection circuit 1j which is the phase difference detection section. The ordinate indicates the phase difference (θV−θI) in a unit of degree, and the abscissa indicates the elapsed time in a unit of msec. Here, “degree” represents a phase difference, and 360 degrees make a period. In the radian unit, one degree is 2 π/360 radian. The abrupt change in the phase difference (θV−θI) shown in FIG. 9 occurs at a width of several msec. When the operator performs coagulation or incision of living tissue in the abdominal cavity of the patient, the coagulation or incision is performed by an operation or gripping in units of several seconds. When coagulation or incision of living tissue is performed too, the impedance is changed when the probe 2b is brought into contact with the living tissue, whereby the phase difference (θV−θI) is also changed. However, the temporal change is in units of seconds, and is not so abrupt a change as shown in FIG. 9. Therefore, when the threshold is determined, it is sufficient if the unit time is several msec to several hundred msec. In order to distinguish the variation in the phase difference (θV−θI) resulting from a crack in the probe, and the variation in the phase difference (θV−θI) resulting from contact of the probe with the living tissue from each other, the inventors have determined a number of thresholds, and have repeated the experiment. As a result of this, by setting the threshold of the variation amount of the phase difference (θV−θI) per 100 msec at 10 degrees, the abnormality detection circuit 1k did not commit any wrong judgment.

Further, as for the time at which the phase difference (θV−θI) is detected, i.e., the time at which the phase difference (θV−θI) is sampled, the instant at which a crack occurs must be accurately grasped. This is because there is the very strong possibility of a probe in which a crack is caused when an ultrasonic wave is applied thereto for a period of several hundred msec to several seconds or longer being broken and falling off, and hence it is necessary to immediately stop or shut down the ultrasonic output. As is apparent from FIG. 9, the crack of the probe 2b has occurred between 100 msec and 115 msec, and hence it is desirable that the detection interval of the phase difference (θV−θI) be 10 msec or less.

### (Effect)

A method is employed in which a threshold determined in advance with respect to a variation amount of the phase difference (θV−θI) per 100 msec is set at 10 degrees, and the probe is judged to be abnormal when the variation amount of the phase difference (θV−θI) exceeds the threshold. By setting the threshold at 10 degrees, the abnormality detection circuit 1k did not commit any wrong judgment. By the threshold setting method, it is possible to accurately and easily distinguish the variation in the phase difference (θV−θI) resulting from an ordinary operation, and the variation in the phase difference (θV−θI) resulting from a crack in the probe 2b from each other.

Further, by setting the interval of sampling of the phase difference (θV−θI) at 10 msec or less, it is possible to grasp the accurate time at which the crack is caused, stop or shut down the ultrasonic output accordingly, and prevent breakage or falling off of the probe greater than the crack.

### Third Embodiment

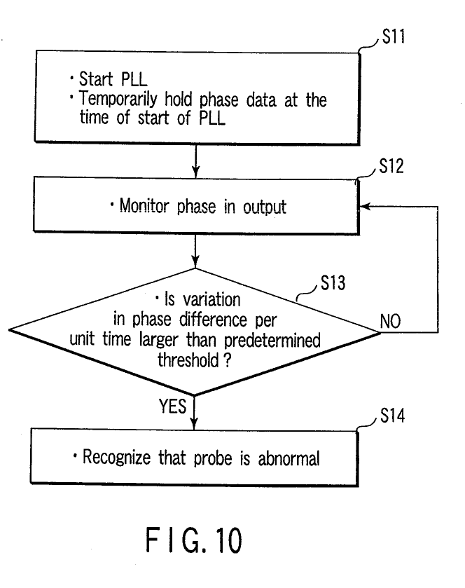

A third embodiment of the present invention will be described below with reference to the block diagram of FIG. 7 and the flowchart of FIG. 10. Here, only the parts different from the first and second embodiments will be described below. Steps S1, and S2 of the flowchart of FIG. 8 correspond to steps S11, and S12 of the flowchart of FIG. 10, and hence detailed description of them will be omitted.

In FIG. 7, a phase difference detection circuit 1j detects a phase difference between an output voltage and an output current from an output voltage/output current detection circuit 1f. This phase difference is varied by a crack in the probe 2b. This is apparent from (B) and (C) in FIG. 6. The phase difference detection circuit 1j compares the variation in the phase difference per unit time with a predetermined threshold (step S13). When the variation in the phase difference is larger than the threshold, an abnormality detection circuit 1k judges that there is an abnormality in the probe (step S14). Further, the abnormality detection circuit 1k can judge the fact that the variation amount of the phase difference is not in the normal value range for a certain period of time, and the phase continues to lead or lag shown in the first embodiment to be abnormal, and add the judgment in an overlapping manner. As a result of this, more accurate judgment can be made. Although the abnormality detection circuit 1k can also judge the probe to be abnormal according to the value of the phase difference, and the variation in the phase difference, the abnormality detection circuit 1k can make the variation in the impedance a condition for judging the probe to be abnormal. This enables a more accurate and more appropriate judgment.

### (Effect)

By comparing the variation amount of the phase difference per unit time with the predetermined threshold, the judgment of the abnormality is made. Furthermore, when the value of the phase difference deviates from the normal value range for a predetermined period of time too, a judgment of the abnormality can be made. By making an abnormality judgment when these conditions (that an amount of the variation in the phase difference exceeds a predetermined threshold and/or that a period of time for which the value of the phase difference deviates from the normal value range exceeds a certain fixed period of time) are satisfied, a more accurate and appropriate judgment can be made, and a more accurate and appropriate stoppage or shutdown of the ultrasonic output can be performed.

### Fourth Embodiment

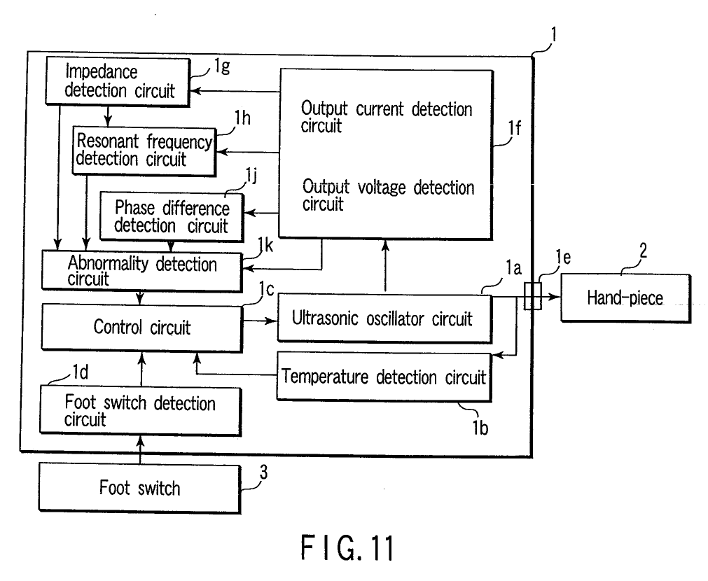

A fourth embodiment will be described below with reference to the block diagram of FIG. 11. This block diagram resembles the block diagram of FIG. 7, and includes a resonant frequency detection circuit 1h, and a temperature detection circuit 1b in addition to the block diagram of FIG. 7. It is known that a resonant frequency detected by the resonant frequency detection circuit 1h varies from fr to fr′ due to a crack in the probe 2b from (B) and (C) in FIG. 6. It is also known that the temperature variation of the hand-piece 2 is due to the crack of the probe 2b by measuring the temperature of the hand-piece 2. More specifically, the capacity of the hand-piece 2 is correlated with the internal temperature thereof, and hence by measuring the capacity thereof the temperature can be measured. Thus, the value of the phase difference, variation amount of the phase difference, variation amount of the resonant frequency and/or the temperature of the hand-piece 2 are compared with the respective thresholds, and when it is judged that any one of the above values is a value larger than the threshold, the probe is judged to be abnormal, whereby the ultrasonic output is stopped or shut down.

### (Effect)

By measuring the resonant frequency or the temperature of the hand-piece 2 in addition to the detection of the phase difference, the crack in the probe can be grasped more accurately and appropriately.

What is claimed is:

1. A power supply apparatus for operation for outputting power to a surgical instrument comprising:
a phase difference detection section for detecting a phase difference between an output voltage and an output current from the power in the output; and
an abnormality detection section for detecting an abnormality according to whether or not a period of time for which the phase difference deviates from a predetermined normal value range exceeds a predetermined period of time.

2. A power supply apparatus for operation for outputting power to a surgical instrument comprising:
a phase difference detection section for detecting a phase difference between an output voltage and an output current from the power in the output; and
an abnormality detection section for detecting an abnormality according to whether or not a variation value of the phase difference per unit time exceeds a predetermined threshold.

3. The power supply apparatus for operation according to claim 1, wherein the predetermined period of time is 100 msec or more.

4. The power supply apparatus for operation according to claim 2, wherein the predetermined threshold per unit time is 10 degrees/100 msec.

5. The power supply apparatus for operation according to claim 1 or 2, wherein when an abnormality is detected by the abnormality detection section, the output of the power to the surgical instrument is stopped.

6. The power supply apparatus for operation according to claim 1 or 2, wherein
the surgical instrument is provided with an ultrasonic vibrator and a probe for transmitting the vibration of the ultrasonic vibrator to a distal end thereof, and
the power to be output is ultrasonic power for driving the ultrasonic vibrator.
